SEQUENCE LISTING (23 sequences)

SEQ ID NO: 1 - protein, 118 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 2 - protein, 119 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 3 - protein, 115 aa, synthetic construct
XXELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVR

SEQ ID NO: 4 - protein, 114 aa, synthetic construct
XXELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVR

SEQ ID NO: 5 - protein, 117 aa, synthetic construct
EEXLQVIQPDKXVXVAAGEXAXLXCTXTSLIPVGPIQWFRGAGPXRELIYNQKEGHFPRVTTVSXXDLTKRXNMDFXIXIXNITPADAGTYYCVKFRKGSPDDXEFKSGAGTELSVR

SEQ ID NO: 6 - protein, 215 aa, synthetic construct
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 224 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 224 aa, synthetic construct
PPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 224 aa, synthetic construct
PPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 475 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASTKGPSVFPLAPEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 470 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 12 - protein, 446 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 226 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 573 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 15 - protein, 226 aa, synthetic construct; identical to SEQ ID NO: 13

SEQ ID NO: 16 - protein, 602 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSASTKGPSVFPLAPEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 17 - protein, 454 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGSEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 18 - protein, 226 aa, synthetic construct; identical to SEQ ID NO: 13

SEQ ID NO: 19 - protein, 115 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 20 - protein, 103 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYG

SEQ ID NO: 21 - protein, 107 aa, synthetic construct [sequence omitted]

SEQ ID NO: 22 - protein, 8 aa, synthetic construct
GGGSGGGS

SEQ ID NO: 23 - protein, 13 aa, synthetic construct
ASTKGPSVFPLAP

(19) United States
(12) Patent Application Publication
(10) Pub. No.: US 2025/0257113 A1
(43) Pub. Date: Aug. 14, 2025

(54) MULTIVALENT SIRP-ALPHA FUSION POLYPEPTIDES

(71) Applicants: The Board of Trustees of the Leland Stanford Junior University, Stanford, CA (US); BITTERROOT BIO, INC., Palo Alto, CA (US)

(72) Inventors: Jens-Peter Volkmer, Palo Alto, CA (US); Jie Liu, Palo Alto, CA (US); Irving L. Weissman, Stanford, CA (US)

(21) Appl. No.: 18/849,291

(22) PCT Filed: Mar. 23, 2023
(86) PCT No.: PCT/US2023/064884

§ 371 Date: Sep. 20, 2024

Related U.S. Application Data
(60) Provisional application No. 63/323,432, filed on Mar. 24, 2022.

(51) Int. Cl. C07K 14/705; A61K 38/00

(52) U.S. Cl. CPC C07K 14/70503; A61K 38/00; C07K 2319/30

(57) ABSTRACT

Provided herein are multivalent signal-regulatory protein α (SIRPα) fusion polypeptides and methods of use thereof. The compositions and methods described herein may be used to treat a variety of diseases or disorders, such as cardiovascular disease.

### CROSS REFERENCE TO RELATED APPLICATIONS

This application claims priority to U.S. Provisional Patent Application No. 63/323,432 filed Mar. 24, 2022, the contents of which are incorporated by reference in their entirety.

### INCORPORATION BY REFERENCE OF SEQUENCE LISTING

The contents of the electronic sequence listing (BTRT_009_01WO_SeqList_ST26.xml; Size: 26,303 bytes; and Date of Creation: Mar. 21, 2023) are herein incorporated by reference in their entirety.

### BACKGROUND

Optimization of protein-based therapies remains a challenge as smaller peptides are cleared quickly from the bloodstream, while larger proteins or antibodies may not be readily taken up by target cells. One example is the regulation of signal-regulatory protein α (SIRPα) engagement with CD47, which is applicable to a wide range of diseases, and which would benefit from the development of improved pharmacokinetics, target engagement, and safety profiles. Therefore, a need exists for optimized signal-regulatory protein α (SIRPα) therapies for the regulation of SIRPα-CD47 engagement. Provided herein are compositions and methods that address this need.

### SUMMARY

The present disclosure provides multivalent signal-regulatory protein α (SIRPα) fusion polypeptides comprising SIRPα domains and Fc domains in a ratio of at least two to one. The multivalent signal-regulatory protein α (SIRPα) fusion polypeptides treatments described herein provide improved pharmacokinetics, target engagement, and safety profiles relative to monovalent SIRPα fusion polypeptides.

### BRIEF DESCRIPTION OF THE DRAWINGS

FIG. 1 shows a schematic representation of a monovalent signal-regulatory protein α (SIRPα) fusion polypeptide, wherein the SIRPα (CV-1) domain and IgG4 Fc domain are in a ratio of one to one.

FIGS. 2A and 2B show schematic representations of exemplary multivalent SIRPα polypeptides of the disclosure wherein the SIRPα (CV-1) domain and IgG4 Fc domain are in a ratio of two to one. FIG. 2A shows the exemplary multivalent configurations of SIRPα fusion polypeptides V1, V2, and V3. FIG. 2B shows the configurations of V4, V5, and V6 multivalent SIRPα polypeptides.

FIG. 3 shows two line-plots that demonstrate the in vitro potency of exemplary multivalent SIRPα fusion polypeptides of the disclosure, of the configuration of V1, V2, and V3, relative to CV1-G4, a CD47 antibody, and a CD20 antibody in phagocytosis assays at high (66 nM), left, and low (0.66 nM), right, concentrations of reagents.

FIG. 4 shows two line-plots that demonstrate the in vitro potency of exemplary multivalent SIRPα fusion polypeptides of the disclosure in combination with a CD20 antibody, rituximab, relative to the potencies of CV1-G4 or a CD47 antibody, in phagocytosis assays at high (66 nM), left, and low (0.66 nM), right, concentrations of reagents.

FIG. 5. shows representative images of the in vitro effects on red blood cell agglutination of exemplary multivalent SIRPα fusion polypeptides of the disclosure, of the configurations of V1, V2, V3, compared to that of CV1-G4, a CD47 antibody, and the control phosphate buffered saline (PBS).

FIG. 6. shows the scoring of images of red blood cell agglutination (e.g., FIG. 5) of exemplary multivalent SIRPα fusion polypeptides with the configuration of V1, V2, V3 of the disclosure, compared to that of CV1-G4, a CD47 antibody, and the control phosphate buffered saline (PBS). N=34 experiments.

FIGS. 7A-7C show the purity analysis of exemplary multivalent SIRPα fusion polypeptides with the configurations of V1, V2, V3 of the disclosure by size-exclusion ultra-performance liquid chromatography elution over time. The number of peaks, the time at which they elute, and the relative percentage of each are shown for each multivalent polypeptide in the table below the graph. FIG. 7A shows data associated with the V1 configuration. FIG. 7B shows data associated with the V2 configuration. FIG. 7C shows data associated with the V3 configuration.

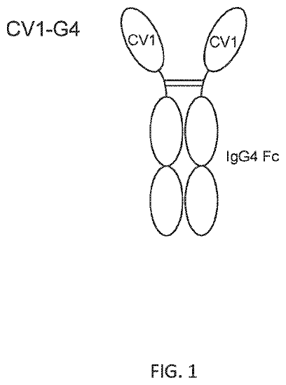

### DETAILED DESCRIPTION

Provided herein are multivalent signal-regulatory protein α (SIRPα) fusion polypeptides useful for the engagement of endogenous SIRPα with CD47. The multivalent SIRPα fusion polypeptides as provided herein comprise SIRPα domains and Fc domains in a ratio of at least two to one. The multivalent fusion polypeptides of the disclosure may be used for the treatment of diseases and disorders including, but not limited to, cardiovascular diseases, fibrosis, cancers, infectious diseases, hematological diseases and disorders, and neurological diseases.

### I. Definitions

Unless otherwise defined herein, scientific and technical terms used herein shall have the meanings that are commonly understood by those of ordinary skill in the art. Generally, nomenclature used in connection with, and techniques of, chemistry, molecular biology, cell biology, immunology, pharmacology, and protein chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “an agent” refers to one or mixtures of such candidates, and reference to “a method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the term “nucleic acid sequence” or “nucleotide sequence” refers to a molecule comprising either of a sequence of DNA or RNA nucleotides, presented from 5′ to 3′.

As used herein, “antibody” includes reference to a full-length immunoglobulin molecule immunologically reactive with a particular antigen, including both polyclonal and monoclonal antibodies. The term includes humanized antibodies, chimeric antibodies e.g., murine variable region with a human constant region, and conjugated antibodies.

The term “Fc domain” as used herein (also interchangeably referred to herein as “Fc sequence”, “Fc region”, or simply as “Fc”) refers to a fragment crystallizable region monomer of an antibody domain comprising a constant heavy chain 2 domain (CH2) and a constant heavy chain 3 domain (CH3). The “Fc domain” sequence can comprise a wild type or modified IgG hinge region sequence. In some embodiments, Fc domains dimerize or form other multimers. Exemplary human Fc domains include IgG1, IgG2, IgG3, and IgG4.

Unless otherwise noted, modifications in an Fc domain are presented according to the EU numbering scheme. However, there are multiple numbering schemes which can be easily cross-referenced to one of skill in the art (www.imgt.org/IMGTScientificChart/Numbering/Hu_IGHGnber.html#refs).

The terms “treatment”, “treating” and the like are used herein to generally mean obtaining a desired pharmacologic and/or physiologic effect with a therapeutic agent. The effect may be prophylactic in terms of completely or partially preventing a disease or symptom thereof, e.g., reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject's treatment will be administered during the symptomatic stage of the disease, and in some embodiments, after the symptomatic stage of the disease.

The terms “individual,” “subject,” and “patient” are used interchangeably herein and refer to any subject for whom treatment is desired. The subject may be a mammalian subject. Mammalian subjects include, e. g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

### II. Signal-Regulatory Protein α (SIRPα) Fusion Polypeptides

The blocking of signal-regulatory protein a (SIRPα) engagement with the CD47 protein allows for the phagocytic engulfment of CD47-expressing cells. Provided herein are multivalent SIRPα fusion polypeptides that may be used to block the engagement of SIRPα with CD47. The multivalent SIRPα fusion polypeptides of the disclosure comprise at least two SIRPα domains and at least one antibody fragment crystallizable region (Fc) domain, wherein the SIRPα domains and the Fc domains are in a ratio of at least two to one.

In some embodiments, a SIRPα fusion polypeptide, comprising at least two SIRPαdomains and at least one Fc domain, associates with at least one other SIRPα polypeptide comprising at least two SIRPα domains and at least one Fc domain, and therein forms a dimer, trimer, tetramer, pentamer, or other multimer. In exemplary embodiments, a SIRPα fusion polypeptide as provided herein forms a dimer. As used herein, the term “SIRPα fusion polypeptide” refers equivalently to a monomer and any multimeric form of a SIRPα fusion polypeptide.

### SIRPα Polypeptide Sequences

A multivalent SIRPα fusion polypeptide as provided herein comprises SIRPα domains and Fc domains in a ratio of at least two to one. In this section the SIRPα domain is described. The SIRPα domains as provided herein comprise the membrane distal (D1) domain of human SIRPα which mediates binding to CD47 of the SIRPα protein.

In some embodiments, a multivalent SIRPα fusion polypeptide comprises a wild type SIRPα D1 sequence comprising SEQ ID NO: 1 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 1
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS

In some embodiments, a multivalent SIRPα fusion polypeptide of the disclosure comprise a SIRPα D1 domain with one or more amino acid changes relative to a wild type sequence of the D1 domain, for example a D1 domain of SEQ ID NO: 1. In some embodiments, a multivalent SIRPα fusion polypeptide comprises a SIRPα D1 domain, with at least one amino acid change relative to the wild-type sequence of the D1 domain. In some embodiments, a multivalent SIRPα fusion polypeptide comprises a SIRPα D1 domain, with at least two amino acid changes relative to the wild-type sequence of the D1 domain. In some embodiments, a multivalent SIRPα fusion polypeptide comprises a SIRPα D1 domain, with at least three amino acid changes relative to the wild-type sequence of the D1 domain. In some embodiments, a multivalent SIRPα fusion polypeptide comprises a SIRPα D1 domain, with at least four amino acid changes relative to the wild-type sequence of the D1 domain. In some embodiments, a multivalent SIRPα fusion polypeptide comprises a SIRPα D1 domain, with at least five amino acid changes relative to the wild-type sequence of the D1 domain. In some embodiments, a multivalent SIRPα fusion polypeptide comprises a SIRPα D1 domain, with at least six amino acid changes relative to the wild-type sequence of the D1 domain. In some embodiments, a multivalent SIRPα fusion polypeptide comprises a SIRPα D1 domain, with at least seven amino acid changes relative to the wild-type sequence of the D1 domain. In some embodiments, a multivalent SIRPα fusion polypeptide comprises a SIRPα D1 domain, with at least eight amino acid changes relative to the wild-type sequence of the D1 domain. In some embodiments, a multivalent SIRPα fusion polypeptide comprises a SIRPα D1 domain, with at least nine amino acid changes relative to the wild-type sequence of the D1 domain. In some embodiments, a multivalent SIRPα fusion polypeptide comprises a SIRPα D1 domain, with at least ten amino acid changes relative to the wild-type sequence of the D1 domain. In some embodiments, a multivalent SIRPα fusion polypeptide comprises a SIRPα D1 domain, with at least eleven amino acid changes relative to the wild-type sequence of the D1 domain. In some embodiments, a multivalent SIRPα fusion polypeptide comprises a SIRPα D1 domain, with at least twelve amino acid changes relative to the wild-type sequence of the D1 domain. In some embodiments, a multivalent SIRPα fusion polypeptide comprises a SIRPα D1 domain, with at least thirteen amino acid changes relative to the wild-type sequence of the D1 domain. In some embodiments, a multivalent SIRPα fusion polypeptide comprises a SIRPα D1 domain, with at least fourteen amino acid changes relative to the wild-type sequence of the D1 domain. In some embodiments, a multivalent SIRPα fusion polypeptide comprises a SIRPα D1 domain, with at least fifteen amino acid changes relative to the wild-type sequence of the D1 domain. In some embodiments, a multivalent SIRPα fusion polypeptide comprises a SIRPα D1 domain, with at least sixteen amino acid changes relative to the wild-type sequence of the D1 domain. In some embodiments, a multivalent SIRPα fusion polypeptide comprises a SIRPα D1 domain, with at least seventeen amino acid changes relative to the wild-type sequence of the D1 domain. In some embodiments, a multivalent SIRPα fusion polypeptide comprises a SIRPα D1 domain, with at least eighteen amino acid changes relative to the wild-type sequence of the D1 domain. In some embodiments, a multivalent SIRPα fusion polypeptide comprises a SIRPα D1 domain, with at least nineteen amino acid changes relative to the wild-type sequence of the D1 domain. In some embodiments, a multivalent SIRPα fusion polypeptide comprises a SIRPα D1 domain, with at least twenty amino acid changes relative to the wild-type sequence of the D1 domain. In some embodiments, a multivalent SIRPα fusion polypeptide comprises a SIRPα D1 domain, with at least twenty-one amino acid changes relative to the wild-type sequence of the D1 domain. In some embodiments, a multivalent SIRPα fusion polypeptide comprises a SIRPα D1 domain, with at least twenty-two amino acid changes relative to the wild-type sequence of the D1 domain. In some embodiments, a multivalent SIRPα fusion polypeptide comprises a SIRPα D1 domain, with at least twenty-three amino acid changes relative to the wild-type sequence of the D1 domain. In some embodiments, a multivalent SIRPα fusion polypeptide comprises a SIRPα D1 domain, with at least twenty-four amino acid changes relative to the wild-type sequence of the D1 domain. In some embodiments, a multivalent SIRPα fusion polypeptide comprises a SIRPα D1 domain, with at least twenty-five amino acid changes relative to the wild-type sequence of the D1 domain.

In some embodiments, a multivalent SIRPα fusion polypeptide that comprises a SIRPα D1 domain, with one or more amino acid changes relative to the wild-type sequence of the D1 domain, exhibits a higher binding affinity (i.e., lower K_{D} value) to CD47 by at least 5-fold, 10-fold, at least 20-fold, at least 50-fold, at least 100-fold, at least 500-fold, at least 1000-fold or more relative to a multivalent SIRPα fusion polypeptide comprising a wild type SIRPα D1 domain.

In some embodiments, a multivalent SIRPα fusion polypeptide of the disclosure comprises a modification relative to the wild type SIRPα D1 domain sequence of SEQ ID NO: 1 at one or more of the following residues V6, S14, S20, I22, H24, V27, I31, A45, E47, K53, E54, H56, S66, E70, S77, V92, and/or a duplication of the D100 residue.

In some embodiments, a multivalent SIRPα fusion polypeptide of the disclosure comprises a modification relative to the wild type SIRPα D1 domain sequence of SEQ ID NO: 1 of one or more of the following: V6I, S14L, S20T, I22T, H24R, V27I, I31F, A45G, E47V, K53R, E54Q, H56P, S66T, E70N, S77R, V92I, and/or a duplication of the D100 residue.

In some embodiments, a multivalent SIRPα fusion polypeptide of the disclosure comprises a SIRPα D1 sequence of SEQ ID NO: 2, referred to herein as CV1 (an exemplary SIRPα polypeptide that exhibits a high binding affinity to CD47), or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 2
EEELQIIQPDKSVLVAAGETATLRCTITSLFPVGPIQWFRGAGPGRVLIY
NQRQGPFPRVTTVSDTTKRNNMDFSIRIGNITPADAGTYYCIKFRKGSPD
DVEFKSGAGTELSVRAKPS

In some embodiments, a multivalent SIRPα fusion polypeptide of the disclosure comprises a SIRPα D1 sequence of SEQ ID NO: 3 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 3
XXELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVR

In some embodiments, a multivalent SIRPα fusion polypeptide of the disclosure comprises a SIRPα D1 sequence of SEQ ID NO: 4 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 4
XXELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVR

In some embodiments, a multivalent SIRPα fusion polypeptide of the disclosure comprises a SIRPα D1 sequence of SEQ ID NO: 5 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 5
EEXLQVIQPDKXVXVAAGEXAXLXCTXTSLIPVGPIQWFRGAGPXRELIY
NQKEGHFPRVTTVSXXDLTKRXNMDFXIXIXNITPADAGTYYCVKFRKGS
PDDXEFKSGAGTELSVR

In some embodiments, a multivalent SIRPα fusion polypeptide of the disclosure comprises one or more of the following mutations relative to the SIRPα D1 sequences of SEQ ID NOS: 1-5: E3G, L4V, L4I, V6I, V6L, S12F, S14L, S20T, A21V, I22T, H24L, H24R, V27A, V27I, V27L, I31F, I31S, I31T, Q37H, A45G, E47V, E47L, K53R, E54Q, E54P, H56P, H56R, V63I, E65D, S66T, S66G, S66L, K68R, E70N, M72R, S75P, R77S, S79G, N80A, N80X, 181N, T82N, P83N, P83X, V92I, F94L, F94V, duplication of D100, E102V, E102T, E102F, F103E, F103V, K104F, K104V, A115G, K116A, and K116G, wherein X=any amino acid.

In some embodiments, a multivalent SIRPα fusion polypeptide sequence of the disclosure may comprise any of the SIRPα D1 sequences described in WO2013109752, WO2014094122A1, WO2017027422, WO2016023040, and WO2016024021A1, incorporated herein by reference in their entirety.

### Fc Domains

The multivalent SIRPα fusion polypeptides provided herein comprise SIRPα domains and Fc domains in a ratio of at least two to one. The Fc domains provided herein can be of any species, e.g., human or mouse, or may be a non-naturally occurring Fc domain, e.g. a human or mouse IgG Fc domain comprising one or more modifications. In some embodiments, the Fc domain is a human IgG1 or IgG4 Fc domain. Canonical sequences for these are presented herein. In some embodiments the Fc domain is a human IgG2 or IgG3 Fc domain, or a mouse IgG1, IgG2a, IgG2b, or IgG3 Fc domain.

In some embodiments, a multivalent SIRPα fusion polypeptide of the disclosure comprises the IgG1 Fc amino sequence of SEQ ID NO: 6 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 6
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK

In some embodiments, a multivalent SIRPα fusion polypeptide of the disclosure comprises the IgG4 Fc amino sequence of SEQ ID NO: 7 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 7
PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGK

In some embodiments, a multivalent SIRPα fusion polypeptide of the disclosure comprises an Fc domain of an IgG4 human Fc domain and the polypeptide is prone to the dynamic process of Fab-arm exchange. Accordingly, in some embodiments the IgG4 Fc domain may comprise a S228P substitution relative to SEQ ID NO: 7 according to EU numbering scheme, resulting in the reduction of this process. In some embodiments, a multivalent SIRPα fusion polypeptide of the disclosure comprises the IgG4 Fc amino sequence of SEQ ID NO: 8 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 8
PPCPPCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGK

In some embodiments, the IgG4Fc amino acid sequence comprises the substitution L445P relative to SEQ ID NO: 8, according to the EU numbering scheme.

In some embodiments, one or more modifications (e.g., substitutions) may be introduced into an IgG Fc sequence to increase engagement with the neonatal Fc receptor (FcRn), and extend the half-life of, e.g., the serum half-life, of the SIRPα fusion polypeptide. In some embodiments, one or more modifications may be introduced in an Fc domain of a multivalent SIRPα fusion polypeptide of the disclosure to: increase FcRn binding; reduce aggregation of the polypeptide; facilitate purification of the polypeptide; increase complement dependent cytotoxicity of the system; increase effector function; decrease effector function; increase half-life; and/or increase endocytosis and degradation of a bound CD47 protein.

In some embodiments, a multivalent SIRPα fusion polypeptide of the disclosure comprises a human IgG1 Fc sequence of SEQ ID NO: 6 comprising one or more modifications (e.g., substitutions) to increase effector function. In some embodiments, the substitutions are selected from the group consisting of V215A, G236A, S239D, 1332E. Exemplary combinations include: G236A-S239D, G236A-I332E, S239D-I332E, V215A-G236A-S239D-I332E, G236A-S239D-I332E, K326W-E333S, S267E-H268F-S324T, and E345R-E430G-S440Y, F243L-R292P-Y300L-V305I-P396L, S239D-I332E, S298A-E333A-K334A, L234Y-L235Q-G236W-S239M-H268D-D270E-S298A, and D270E-K326D-A330M-K334E, relative to SEQ ID NO: 6 according to the EU numbering scheme.

In some embodiments, a multivalent SIRPα fusion polypeptide of the disclosure comprises a human IgG1 Fc sequence of SEQ ID NO: 6 comprising one or more modifications (e.g., substitutions) to decrease effector function. In some embodiments, the substitutions are selected from the group consisting of: N297A, N297Q, N297G, L235E, L234A, L235A, K214R, P329G, D356E, and L358M, according to the EU numbering scheme.

In some embodiments, a multivalent SIRPα fusion polypeptide of the disclosure comprises a human IgG4 Fc sequence of SEQ ID NOS: 7 or 8 comprising one or more modifications (e.g., substitutions) to decrease effector function. In some embodiments, the substitutions are selected from the group consisting of: L235A, L235E, S228P, and F234A, according to the EU numbering scheme. Exemplary combinations include L235E-S228P, S228P-F234A, and S228P-F234A-L235A.

In other embodiments, a multivalent SIRPα fusion polypeptide of the disclosure comprises an IgG1 Fc or an IgG4 Fc domain in which a modification is present to increase serum half-life. In some embodiments the mutations are selected from the group consisting of T250Q, M252Y, S254T, T256E, S267E, N325S, L328F, N343S, M428L, N434F, and H443K relative to IgG1 Fc domain sequence SEQ ID NO: 6 or IgG4 Fc domain sequence SEQ ID NOS: 7 or 8 according to the EU numbering scheme. In some embodiments the mutations are selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme.

In some embodiments, a SIRPα fusion polypeptide of the disclosure comprises an Fc domain of an IgG4 human Fc domain (e.g. SEQ ID NOS: 7 or 8) with the substitutions of M252Y, S254T, and T256E relative to SEQ ID NOS: 7 or 8, according to the EU numbering scheme.

In some embodiments, a SIRPα fusion polypeptide of the disclosure comprises the human IgG4 Fc amino sequence of SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 9
PPCPPCPAPEFLGGPSVFLFPPKPKDTLYITREPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGK

### SIRPα Fusion Polypeptide Valency

The present disclosure provides multivalent signal-regulatory protein α (SIRPα SIRPα) fusion polypeptides comprising SIRPα domains and Fc domains in a ratio of at least two to one. The multivalent signal-regulatory protein α (SIRPα) fusion polypeptides described herein provide improved characteristics relative to monovalent SIRPα fusion polypeptides, including but not limited to improved pharmacokinetics, target engagement, and safety profiles relative to monovalent SIRPα fusion polypeptides.

As contemplated herein, a monovalent SIRPα fusion polypeptide comprises SIRPα domains and Fc domains in a ratio of 1:1.

As provided herein, a multivalent SIRPα fusion polypeptide of the disclosure comprises SIRPα domains and Fc domains in a ratio of a least 2:1, 3:1, 4:1, 5:1, 6:1, 7:1 or 8:1. In some embodiments, the multivalent SIRPα fusion polypeptides comprise SIRPα and Fc domains in a ratio of 5:2, 7:2, 9:2, 11:2, or 13:2. In some embodiments, a multivalent SIRPα fusion polypeptide comprises SIRPα domains and Fc domains in a ratio of 2:1.

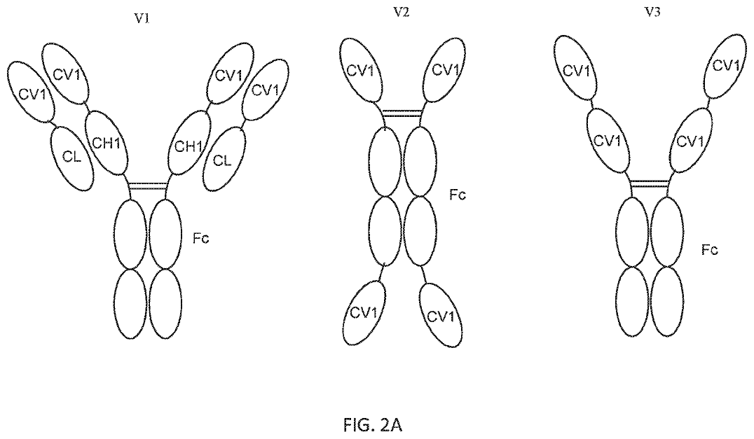

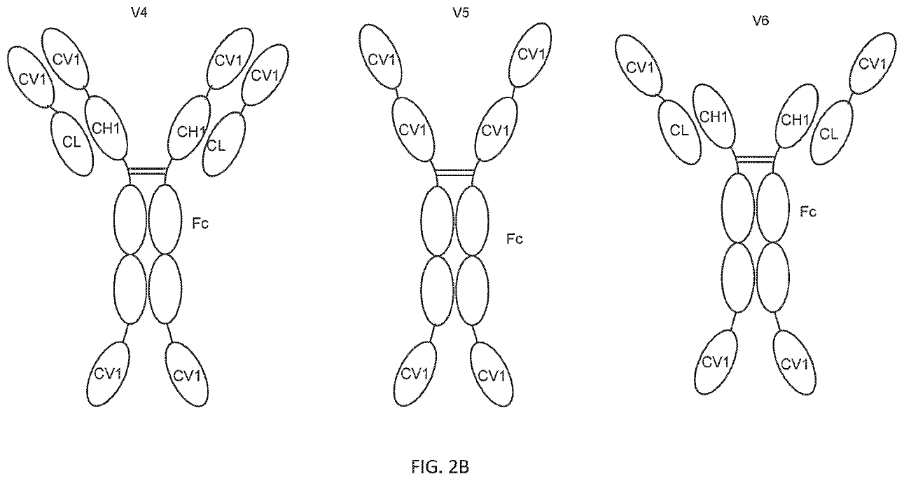

In some embodiments, the multivalent SIRPα fusion polypeptide of the disclosure comprises at least two SIRPα domains at the N terminus of the Fc domain. In some embodiments, the multivalent SIRPα fusion polypeptide comprises at least three, at least four, at least five, at least six, at least seven, or at least eight SIRPα domains at the N terminus of the Fc domain. Exemplary structures are provided in FIGS. 2A and 2B.

In some embodiments, the multivalent SIRPα fusion polypeptide comprises at least two SIRPα domains at the C terminus of a Fc domain. In some embodiments, the multivalent SIRPα fusion polypeptide comprises at least three, at least four, at least five, or at least six, at least seven, or at least eight SIRPα domains at the C terminus of an Fc domain.

In some embodiments, the multivalent SIRPα fusion polypeptide comprises at least two SIRPα domains, wherein at least one is positioned at the C terminus and at least one SIRPα domain is positioned at the N terminus of the Fc domain. In some embodiments, the multivalent SIRPα fusion polypeptide comprises an equal number of SIRPα domains at the C terminus and the N terminus of the Fc domain. In some embodiments, the multivalent SIRPα fusion polypeptide comprises an unequal number of SIRPα domains at the C terminus and the N terminus of the Fc domain. Exemplary structures and contemplated configurations are provided in FIGS. 2A and 2B.

In some embodiments, the multivalent SIRPα fusion polypeptide comprises one or more of a constant heavy chain 1 (CH1) of an IgG1 protein. In some embodiments, a SIRPα fusion polypeptide of the disclosure comprises a CH1 sequence of SEQ ID NO: 19 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 19
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAP

In some embodiments, the multivalent SIRPα fusion polypeptide comprises one or more of a constant heavy chain 1 (CH1) of an IgG4 protein. In some embodiments, a SIRPα fusion polypeptide of the disclosure comprises a CH1 sequence of SEQ ID NO: 20 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 20
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYG

In some embodiments the one or more CH1 is at the N terminus of the Fc domain. In some embodiments the one or more constant CH1 is at the C terminus of the Fc domain. Exemplary structures are provided in FIGS. 2A and 2B.

In some embodiments, the multivalent SIRPα fusion polypeptide comprises one or more constant light chains (CL) of an antibody. In some embodiments, a SIRPα fusion polypeptide of the disclosure comprises a CL sequence of SEQ ID NO: 21 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 21
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments the one or more CL is at the N terminus of the Fc domain. In some embodiments the one or more CL is at the C terminus of the Fc domain. Exemplary structures are provided in FIGS. 2A and 2B.

In some embodiments, the multivalent SIRPα fusion polypeptide comprises one or more variable light chains (VL) of an antibody. In some embodiments, the multivalent SIRPα fusion polypeptide comprises one or more variable heavy chains (VH) of an antibody.

In some embodiments, the multivalent SIRPα fusion polypeptide comprises one or more hinge domains.

In some embodiments, a multivalent SIRPα fusion polypeptide comprises a peptide linker. In some embodiments, the multivalent SIRPα fusion polypeptide comprises a peptide linker of about 6, about 7, about 8, about 9, about 10, about 11, about 12, about 13, about 14, about 15, or about 16 amino acids in length. In some embodiments, the peptide linker comprises Glycine (G) and/or Serine(S) amino acids. In some embodiments, the peptide linker is 8 amino acids of G and S amino acids. In some embodiments, the linker is GGGSGGGS. In some embodiments, the linker comprises a human IgG sequence, e.g., ASTKGPSVFPLAP.

In some embodiments, a multivalent SIRPα fusion polypeptide monomer comprises two or more SIRPα domains in tandem, and optionally comprises a linker between the two or more SIRPα domains. In some embodiments, the multivalent SIRPα fusion polypeptide monomer comprises a linker between a SIRPα domain and an antibody constant domain (e.g., an Fc domain, a CH1, or a CL domain). In some embodiments, the multivalent SIRPα fusion polypeptide monomer comprises a linker between a SIRPα domain and an antibody variable domain (e.g. a VL or VH domain). In some embodiments, the multivalent SIRPα fusion polypeptide monomer comprises a linker between an antibody constant domain (e.g., an Fc domain, a CH1, or CL domain) and an antibody variable domain (e.g. a VL or VH domain). Any of the above multivalent SIRPα fusion polypeptide monomers may also dimerize or form multimeric structures.

Exemplary multivalent SIRPα fusion polypeptides include a SIRPα sequence of any of SEQ ID NOS: 1-5, or an amino acid sequence with at least 70%, at least 75%, at least 80%, at least 85%, at least 90%, at least 95%, at least 96%, at least 97%, at least 98%, or at least 99% sequence identity thereto, an Fc sequence of any of SEQ ID NOS: 6-9, or an amino acid sequence with at least 70%, at least 75%, at least 80%, at least 85%, at least 90%, at least 95%, at least 96%, at least 97%, at least 98%, or at least 99% sequence identity thereto. Exemplary multivalent SIRPα fusion polypeptides may also comprise one or more of a CH1 sequence of SEQ ID NOS: 19-20 and a CL sequence of SEQ ID NO: 21, or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises both heavy and light chain sequences, wherein the light chain comprises a SIRPα sequence of SEQ ID NO: 2 and a CL sequence of SEQ ID NO: 21, and the heavy chain comprises a SIRPα sequence of SEQ ID NO: 2, a CH1 of SEQ ID NO: 20, and an Fc domain of SEQ ID NOS: 8 or 9. In some embodiments, a multivalent SIRPα fusion polypeptides of the disclosure comprises SEQ ID NO: 12 or an amino acid sequence with at least 70%, at least 75%, at least 80%, at least 85%, at least 90%, at least 95%, at least 96%, at least 97%, at least 98%, or at least 99% sequence identity thereto, and SEQ ID NO: 13 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises a SIRPα sequence of SEQ ID NO: 2 at both the N terminus and the C terminus of an Fc domain of SEQ ID NOS: 8 or 9. In some embodiments, a multivalent SIRPα fusion polypeptides of the disclosure comprises SEQ ID NO: 11 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises two SIRPα sequences of SEQ ID NO: 2 at the N terminus of an Fc domain of SEQ ID NOS: 8 or 9. In some embodiments, a multivalent SIRPα fusion polypeptides of the disclosure comprises SEQ ID NO: 10 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises both heavy and light chain sequences, wherein the light chain comprises a SIRPα sequence of SEQ ID NO: 2 and a CL sequence of SEQ ID NO: 21, and the heavy chain comprises a CH1 of SEQ ID NO: 20, and a SIRPα sequence of SEQ ID NO: 2 at both the N and C terminus of an Fc domain of SEQ ID NOS: 8 or 9. In some embodiments, a multivalent SIRPα fusion polypeptides of the disclosure comprises SEQ ID NO: 14 or an amino acid sequence with at least 70%, at least 75%, at least 80%, at least 85%, at least 90%, at least 95%, at least 96%, at least 97%, at least 98%, or at least 99% sequence identity thereto, and SEQ ID NO: 15 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises two SIRPα sequences of SEQ ID NO: 2 at the N terminus and one SIRPα sequence of SEQ ID NO: 2 at the C terminus of an Fc domain of SEQ ID NOS: 8 or 9. In some embodiments, a multivalent SIRPα fusion polypeptides of the disclosure comprises SEQ ID NO: 16 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises both heavy and light chain sequences, wherein the light chain comprises a SIRPα sequence of SEQ ID NO: 2 and a CL sequence of SEQ ID NO: 21, and the heavy chain comprises a CH1 of SEQ ID NO: 20 at the N terminus, and a SIRPα sequence of SEQ ID NO: 2 at the C terminus of an Fc domain of SEQ ID NOS: 8 or 9. In some embodiments, a multivalent SIRPα fusion polypeptides of the disclosure comprises SEQ ID NO: 17 or an amino acid sequence with at least 70%, at least 75%, at least 80%, at least 85%, at least 90%, at least 95%, at least 96%, at least 97%, at least 98%, or at least 99% sequence identity thereto, and SEQ ID NO: 18 or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide comprises one or more of SEQ ID NOS: 10-18, or an amino acid sequence with at least 70%, at least 75%, at least 80%, at least 85%, at least 90%, at least 95%, at least 96%, at least 97%, at least 98%, or at least 99% sequence identity thereto.

In some embodiments, a multivalent SIRPα fusion polypeptide is conjugated to a fluorophore, a radionucleotide, a toxin, and/or a diagnostic conjugate or moiety.

### III. Methods of Making Multivalent SIRPα Fusion Polypeptides

Also provided herein are polynucleotides encoding the multivalent SIRPα fusion polypeptides of the disclosure. In some embodiments, the polynucleotide encodes a polypeptide comprising SIRPα domains and Fc domains in a ratio of 2:1 or 3:1. In some embodiments, the polynucleotide encodes any of the aforementioned multivalent SIRPα fusion polypeptides, or a sequence with at least 70%, at least 75%, at least 80%, at least 85%, at least 90%, at least 95%, at least 96%, at least 97%, at least 98%, or at least 99% sequence identity thereto.

In some embodiments, a polynucleotide encoding a multivalent SIRPα fusion polypeptide of the disclosure is introduced (e.g., transfected or transformed) and expressed in a human cell line, or a bacterial cell line. Exemplary cell lines available for production include, but are not limited to, Expi 293 and CHO cell lines.

### IV. Potency and Safety of Multivalent SIRPα Fusion Polypeptides

The multivalent SIRPα fusion polypeptides provided herein, comprising SIRPα domains and Fc domains in a ratio of at least two to one, exhibit improved effects relative to monovalent SIRPα fusion polypeptides, comprising the same SIRPα sequences, wherein the SIRPα domains and Fc domains are present in a ratio of one to one. In some embodiments, a multivalent SIRPα fusion polypeptide has a higher in vivo and/or in vitro potency relative to a monovalent SIRPα fusion polypeptide. In some embodiments, a multivalent SIRPα fusion polypeptide provides surprisingly similar safety profiles to a monovalent SIRPα fusion polypeptide, e.g., wherein RBC agglutination does not increase due to multivalency.

In some embodiments, multivalent SIRPα fusion polypeptides of the disclosure have a higher binding affinity and/or avidity, to a CD47 protein relative to monovalent SIRPα fusion polypeptide comprising the same SIRPα sequences.

In some embodiments, a multivalent SIRPα fusion polypeptide of the disclosure binds a human CD47 protein. In some embodiments, a multivalent SIRPα fusion polypeptide binds both mouse and human CD47 proteins. In some embodiments, a multivalent SIRPα fusion polypeptide binds both human and non-human primate CD47 proteins, e.g., a cynomolgus monkey CD47 protein.

In some embodiments, a multivalent SIRPα fusion polypeptide, comprising SIRPα domains and Fc domains in a ratio of at least two to one, has a higher binding strength to a cell expressing CD47 protein than does a monovalent SIRPα fusion polypeptide comprising the same SIRPα sequences. In some embodiments, the binding strength of a multivalent SIRPα fusion polypeptide to a cell is increased about 1.5×, about 2×, about 3×, about 4×, about 5×, about 6×, about 7×, about 8×, about 9×, about 10×, about 20×, about 50×, about 100×, about 1000×, or about 10,000×relative to that of a monovalent SIRPα fusion polypeptide.

In some embodiments, a multivalent SIRPα fusion polypeptide has a slower blood clearance relative to a monovalent SIRPα fusion polypeptide comprising the same SIRPα sequences. In some embodiments, the blood clearance of a multivalent SIRPα fusion polypeptide is cleared from the blood in about 1.5×, about 2×, about 3×, about 4×, about 5×, about 6×, about 7×, about 8×, about 9×, about 10×, about 20×, about 50×, about 100×, or about 1000×, the time of clearance of a monovalent SIRPα fusion polypeptide.

In some embodiments, a multivalent SIRPα fusion polypeptide is effective at a lower dose in a subject relative to a monovalent SIRPα fusion polypeptide comprising the same SIRPα sequences.

In some embodiments, a multivalent SIRPα fusion polypeptide is effective at a lower dosage frequency relative to a monovalent SIRPα fusion polypeptide comprising the same SIRPα sequences.

In some embodiments, a multivalent SIRPα fusion polypeptide is associated with increased phagocytosis in vivo. In some embodiments, a multivalent SIRPα fusion polypeptide is associated with increased phagocytosis in vitro in a phagocytosis assay relative to a monovalent SIRPα fusion polypeptide comprising the same SIRPα sequences. In some embodiments, the phagocytic events associated with a multivalent SIRPα fusion polypeptide are increased about 1.5×, about 2×, about 3×, about 4×, about 5×, about 6×, about 7×, about 8×, about 9×, about 10×, about 20×, about 50×, about 100×, or about 1000×, that of a monovalent SIRPα fusion polypeptide.

In some embodiments, a multivalent SIRPα fusion polypeptide is associated with comparable red blood cell in vitro agglutination relative to a monovalent SIRPα fusion polypeptide comprising the same SIRPα sequences.

In some embodiments, a multivalent SIRPα fusion polypeptide is associated with comparable expression purity relative to a monovalent SIRPα fusion polypeptide comprising the same SIRPα sequences.

### V. Methods of Using Multivalent SIRPα Fusion Polypeptides

In some embodiments, a multivalent SIRPα fusion polypeptide is administered to a subject in need thereof as a therapeutic. In some embodiments, a multivalent SIRPα fusion polypeptide is administered to treat, for example, a cardiovascular disease, a cancer, fibrosis, an infectious disease, a hematological disease, or a neurological disease.

In some embodiments, a subject selected for treatment with a multivalent SIRPα fusion polypeptide of the disclosure has a cardiovascular disease, and has or is determined to be at risk of having, one or more of atherosclerosis, heart failure, myocardial infarction, cardiomyopathy, acute coronary syndrome, myocarditis, cardiac remodeling, hypertension, angina, restenosis, stroke, aneurysms, thrombosis, phlebitis, peripheral vascular disease, pulmonary arterial hypertension, and autoimmune vasculitis.

In some embodiments, a subject selected for treatment with a multivalent SIRPα fusion polypeptide of the disclosure has a cancer. In some embodiments, a multivalent SIRPα fusion polypeptide of the disclosure amy treat tumor growth and/or tumor metastasis, e.g., of a lymphoma, leukemia, carcinoma, melanoma, glioblastoma, sarcoma, or myeloma.

In some embodiments, a subject selected for treatment with a multivalent SIRPα fusion polypeptide of the disclosure has fibrosis or a fibrotic disease, e.g., a liver or lung fibrotic disease. In some embodiments, the subject has or is at risk of having end-stage liver disease, kidney disease, idiopathic pulmonary fibrosis (IPF), retinal fibrosis, chronic graft rejection from progressive myopathy, or heart failure from cardiac fibrosis.

In some embodiments, a subject selected for treatment with a multivalent SIRPα fusion polypeptide has an infectious disease associated with a virus, bacteria, or fungal pathogen. In some embodiments the subject has a viral infections, e.g., an infection associated with one of a retrovirus, lentivirus, hepadna virus, herpes virus, pox virus, or human papilloma virus. In some embodiments, the subject has an intracellular bacterial infections, e.g., an infection associated with one of Mycobacterium, Chlamydophila, Ehrlichia, Rickettsia, Brucella, Legionella, Francisella, Listeria, Coxiella, Neisseria, Salmonella, or Yersinia species. In some embodiments, the subject has an intracellular protozoan pathogen infection, e.g., an infection associated with one of a Plasmodium species, Trypanosoma species, Giardia species, Toxoplasma species, or Leishmania species.

In some embodiments, a subject is selected for treatment with a multivalent SIRPα fusion polypeptide of the disclosure with a hematological disease or disorder, e.g. a genetic blood disorder or severe combined immunodeficiency. In some embodiments, a multivalent SIRPα fusion polypeptide of the disclosure may be used alone or in combination with other agents to facilitate engraftment of endogenous stem cells prior to hematopoietic stem cell transplant.

In some embodiments, a subject selected for treatment with a multivalent SIRPα fusion polypeptide has a neurological disease.

In some embodiments, a multivalent SIRPα fusion polypeptide may be delivered to a subject in need thereof subcutaneously, intravenously, intravitreally, orally, intranasally, transdermaly, intraperitoneally, intramuscularly, intrathecally, intrapulmonary, vaginally, or rectally. In some embodiments, the multivalent SIRPα fusion polypeptide is administered subcutaneously.

In some embodiments, a multivalent SIRPα fusion polypeptide is conjugated to a fluorophore, a radionucleotide or other imaging or diagnostic moiety. In some embodiments, a multivalent SIRPα fusion polypeptide is administered to a cell or an organism to image the position or concentration of a CD47 protein. In some embodiments, a multivalent SIRPα fusion polypeptide is administered to a cell or an organism to diagnose a disease.

In some embodiments, a multivalent SIRPα fusion polypeptide comprises a conjugated toxin to deliver the toxin to a cell expressing CD47.

In some embodiments, a multivalent SIRPα fusion polypeptide is administered in combination with a CD20 and/or a CD47 antibody, or fragment thereof. dec

### VI. Kits

A multivalent SIRPα fusion polypeptide as described herein may also comprise a therapeutic or diagnostic kit for administration by a medical professional or the subject in need thereof. The kit may comprise for example, a container, a dose of a multivalent SIRPα fusion polypeptide, a syringe and/or a vial, and instructions for use thereof. In some embodiments the kit comprises a multivalent SIRPα fusion polypeptide and instructions for administering the polypeptide to treat a disease.

In some embodiments, the multivalent SIRPα fusion polypeptide of a kit is conjugated to a fluorophore, a radionucleotide or other diagnostic moiety.

### EXAMPLES

### Example 1: Materials and Methods

### Production of Multivalent SIRPα V1, V2, and V3 Fusion Polypeptides

The multivalent SIRPα fusion polypeptides provided herein comprise SIRPα domains and Fc domains in a ratio of at least two to one (as shown in the exemplary constructs of FIGS. 2A and 2B). DNA sequences encoding the CV1 variant of the signal-regulatory protein-α (SIRPα) D1 domain, antibody constant domains (constant heavy chain 3 (CH3), constant heavy chain 2 (CH2), constant heavy chain 1 (CH1), and constant light chain (CL)), were synthesized and cloned into an expression vector. Linkers including GGGSGGGS and ASTKGPSVFPLAP were encoded between the domains. Transfections of the SIRPα fusion polypeptide in the configurations of V1, V2, and V3 were performed in Expi 293 and CHO cells. The culture supernatant was then applied to protein A Sepharose columns (GE Healthcare™). The column was washed with PBS, and the proteins were eluted with eluting buffer (0.1 M sodium citrate buffer, pH 3.0). Collected fractions were neutralized with 1 M Tris pH 9.0. Finally, purified samples were dialyzed against PBS. Purity of the eluted protein fraction was analyzed by sodium dodecyl sulfate polyacrylamide gel electrophoresis (SDS-PAGE) on 10% gels under reducing and non-reducing conditions. Bands were visualized by Coomassie brilliant blue staining (data not shown).

### Size-Exclusion Ultra-Performance Liquid Chromatography of V1, V2, and V3 SIRPα Polypeptides

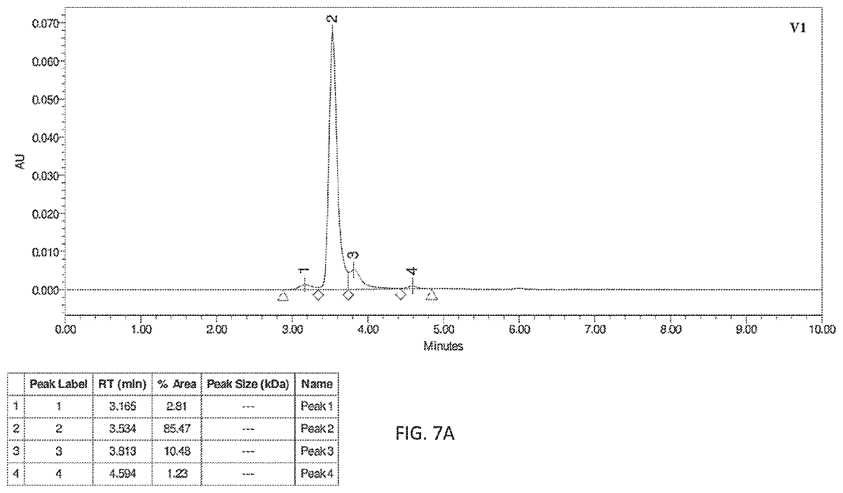

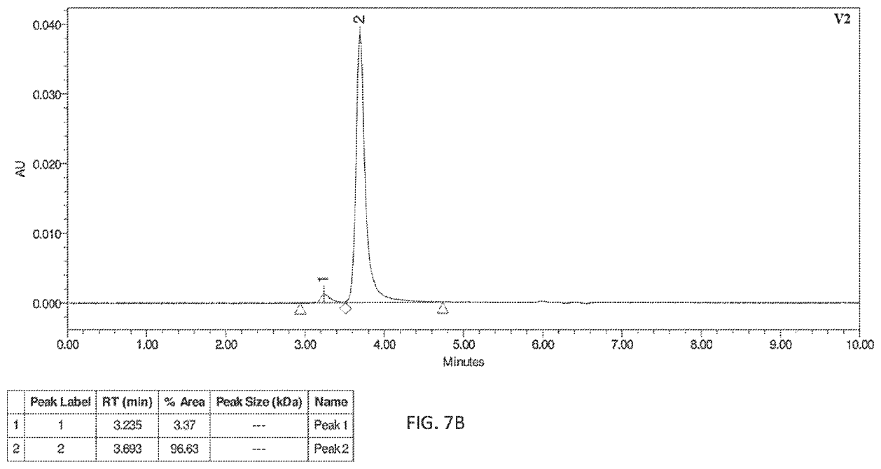

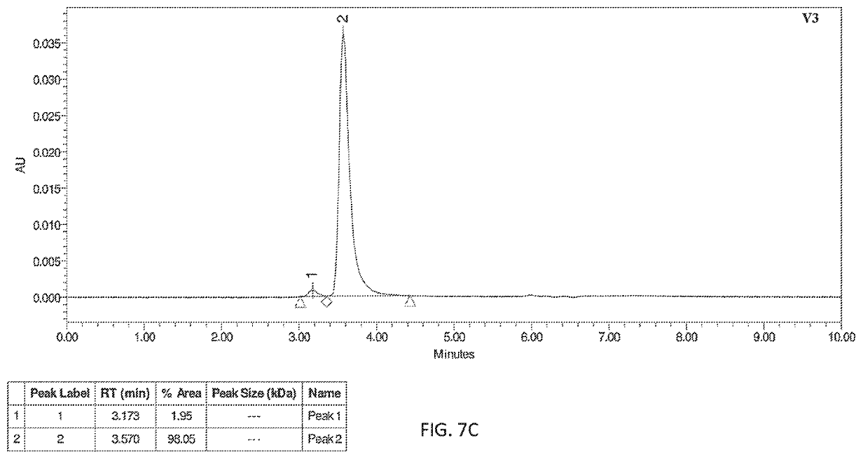

About a 2 μL sample of purified SIRPα polypeptides in the V1, V2, and V3 configurations was injected into a ACQUITY UPLC Protein BEH SEC 200™ 1.7 μm, 4.6×150 mm column with a flow rate of 0.3 mL-min for 10 minutes. A mobile phase with a buffer comprising 50 mM Sodium Phosphate, 500 mM NaCl, pH 6.2 was used. As shown in FIGS. 7A-7C, the V1, V2, and V3 configurations showed 85.47%, 96.63%, and 98.05% purity, respectively, indicating no disordered or mis-folded protein aggregates.

### Measurement of V1, V2, and V3 Configuration Multivalent SIRPα Fusion Polypeptides Binding Affinities to CD47

Binding affinities of V1, V2, and V3 configuration multivalent SIRPα fusion polypeptide dimers to human CD47 and mouse CD47 proteins was performed on a Biacore™ 3000 at 25° C. as follows. The capture polypeptide was immobilized to the desired response unit density (RU) by using 1-Ethyl-3-(3-dimethylaminopropyl) carbodiimide-N-Hydroxysuccinimide (EDC-NHS) amine coupling chemistry as per the GE™ manufacturer protocol. A blank flow cell without a capture polypeptide was used as a control. Un-occupied sites were quenched with 1M ethanol amine. Analyte was then flowed over the chip to test any non-specific binding to the capture polypeptide. Ligand capture binding kinetics were monitored and analyzed using BIAeval software (Biacore) using reference-subtracted values. The binding affinities (i.e. K_{D}) were determined by applying either local or global kinetics using the 1:1 Langmuir fitting model.

### In Vitro Phagocytosis Potency Assays

Macrophages differentiated from healthy human peripheral blood mononuclear cells (PBMC) were harvested with TrypLE, counted, and plated in a density of 50,000 cells per well into 96-well plates. B cell lymphoma Raji cancer cells were rinsed, counted, incubated with pHRodo dye at 37° C. for 30 mins, rinsed again, and centrifuged. The cancer cells were then resuspended and dimerized V1, V2, V3 configuration SIRPα fusion polypeptides, CV1-G4, as well as rituximab, and a CD47 antibody were added to the suspension mixture in concentrations of 66 nM and 0.66 nM. The resulting suspension was plated at a density of 25,000 cells per well on the macrophage containing plates. The plates were then incubated in an IncuCyte™ machine and image scanning was performed every hour for a total of 12 hours.

### In Vitro Human Red Blood Cell (RBC) Agglutination Assay

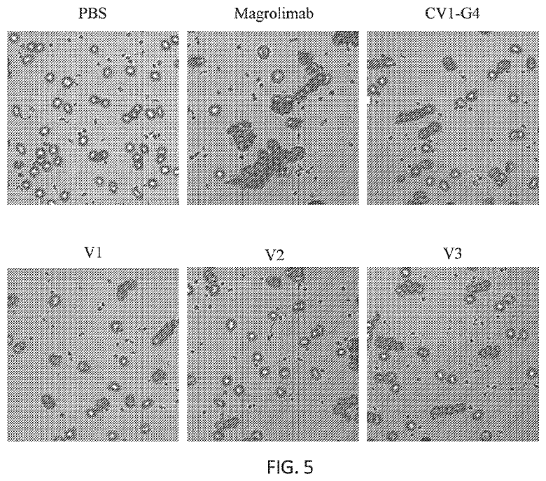

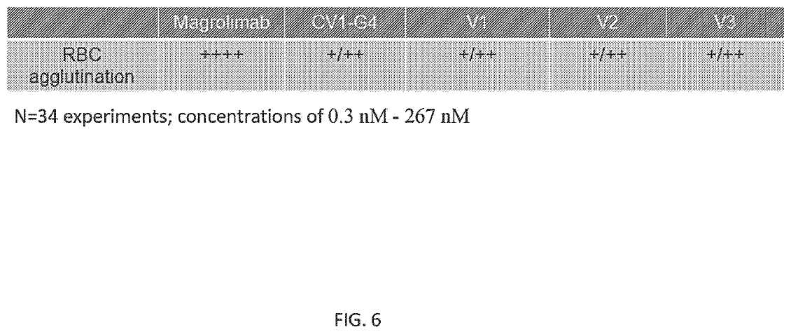

200 μL of human blood was added to each well of a 96-well plate. Dimerized V1, V2, V3 configuration SIRPα fusion polypeptides, CV1-G4, as well as a CD47 antibody were then added, each at concentrations varying from 0.3 nM-267 nM. The experiments were performed N=34 times, with consistent results. The plates were then incubated at 37° C. for 2 hours to allow for agglutination. Drops of blood from each sample were stained by Giemsa and viewed under the microscope (see FIG. 5). Agglutination was characterized as typically performed in the art (Cho et al., Measurement of RBC agglutination with microscopic cell image analysis in a microchannel chip. Clinical Hemorheology and Microcirculation. 2014; 56(1):67-74), on a 0-4+ scale, with 0 representing no reaction, and 4+ indicating a very strong reaction (i.e., where 4+ is a solid clump of red cells and 1+ is the presence of small clumps) (see FIG. 6).

### Example 2: The Multivalent SIRPα Fusion Polypeptides V1, V2, and V3 Bind Both Human and Mouse CD47 Proteins

To improve the efficacy of the signal-regulatory protein-α (SIRPα) CV1-IgG4 Fc fusion variant (CV1-G4) that exhibits high affinity binding to CD47, multivalent SIRPα fusion polypeptides were generated comprising CV1 SIRPα D1 domains and IgG4 Fc domains in a ratio of two to one (see FIG. 2A). (Also contemplated are the multivalent SIRPα fusion polypeptides configurations of V4, V5, and V6, see FIG. 2B.) As shown in FIG. 2A, the multivalent SIRPα fusion polypeptides V1, V2, and V3 configurations each have two CV1 domains for each Fc domain, creating a multivalent polypeptide useful for high affinity binding to CD47.

Binding affinities of multivalent SIRPα fusion polypeptides (in the V1, V2, and V3 configurations) to CD47 were measured. As shown in Table 1, V1, V2, and V3 configurations bind human CD47 with an affinity of 3.7E-11 M, 8.63E-11 M, and 4.48E-11 M, respectively. Notably, unlike an anti-human CD47 monoclonal antibody that does not recognize the mouse CD47 protein, the multivalent SIRPα fusion polypeptides V1, V2, and V3 configurations bind to mouse CD47. Thus, the binding activities and properties of multivalent SIRPα fusion polypeptides V1, V2, and V3 configurations to the mouse CD47 protein allow pre-clinical pharmacology and toxicology studies in mouse models.

TABLE 1
Binding affinity of multivalent SIRPα fusion polypeptide
V1, V2, and V3 configurations to human and mouse CD47 proteins

| | Antigen | Ka (1-Ms) | Kd (1-s) | KD (M) |
|---|---|---|---|---|
| V1 | Human CD47 | 2.79E+06 | 1.03E−04 | 3.70E−11 |
| V3 | Human CD47 | 2.16E+06 | 9.68E−05 | 4.48E−11 |
| V2 | Human CD47 | 1.76E+06 | 1.52E−04 | 8.63E−11 |
| V1 | Mouse CD47 | 5.76E+05 | 0.0137 | 2.37E−08 |
| V3 | Mouse CD47 | 4.08E+05 | 0.0115 | 2.83E−08 |
| V2 | Mouse CD47 | 3.45E+05 | 0.0134 | 3.88E−08 |

### Example 3: The Multivalent SIRPα Fusion Polypeptides V1, V2, and V3 Configurations Demonstrate Increased Potency in in Vitro Phagocytosis

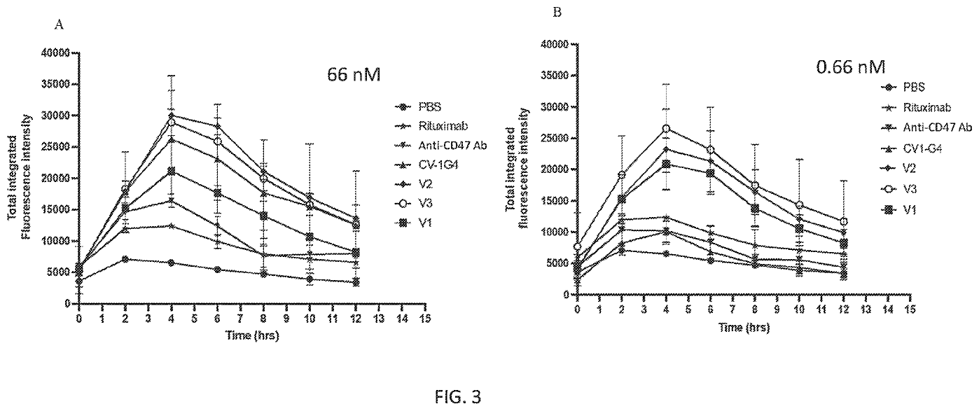

Multivalency provides an opportunity to improve the functional affinity of polypeptides through the combined binding strength of multiple binding domains (i.e., avidity), and in turn, translate into greater potency and efficacy. To test the potency of the V1, V2, and V3 configuration dimerized multivalent polypeptides in cells, in vitro phagocytosis was performed using human macrophages and B cell lymphoma Raji cells. At a concentration of 66.6 nM, V1, V2, and V3 configuration polypeptides induced phagocytic activities comparable to that of dimerized CV1-G4 (FIG. 3, left). Strikingly, when the dose was lowered to 0.66 nM, V1, V2, and V3 configuration polypeptides could still trigger significant phagocytic activities (FIG. 3, right). In contrast, CV1-G4 and a CD47 antibody only had marginal effects on phagocytosis.

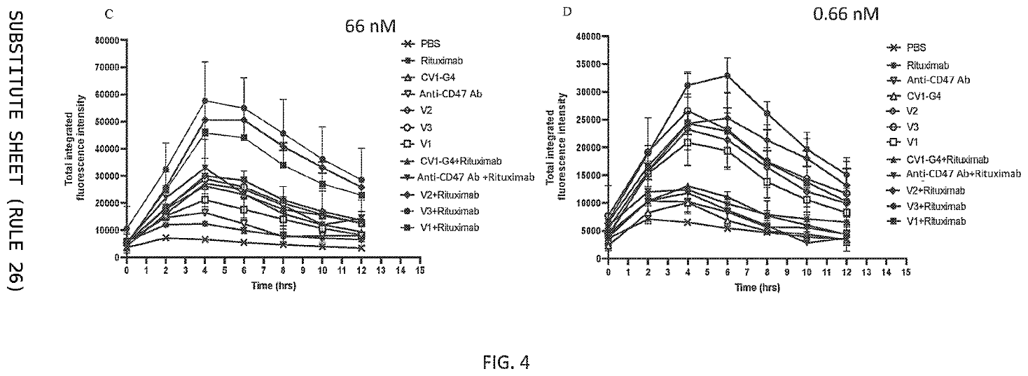

Next, dimerized V1, V2, and V3 configuration polypeptides were tested in combination with rituximab in phagocytosis assays. At a concentration of 66.6 nM, the V1, V2, and V3 polypeptides in combination with rituximab exhibited synergistic effects to promote phagocytosis of Raji cells. These synergistic effects were significantly higher in the V1, V2, and V3 configuration polypeptides, than in the combination of CV1-G4 with rituximab or a CD47 antibody with rituximab (FIG. 4, left). Importantly, V1, V2, and V3 configurations exhibited synergistic effects with rituximab to promote phagocytosis of Raji cells at the lower dose of 0.66 nM, while there was no synergistic effects of CV1-G4 or a CD47 antibody at 0.66 nM in combination with rituximab (FIG. 4, right). Thus, the higher avidity, multivalent SIRPα fusion polypeptides V1, V2, and V3 configurations exhibited greater potency and efficacy than CV1-G4 and anti-CD47 antibodies.

### Example 4: The Multivalent SIRPα Fusion Polypeptides V1, V2, and V3 Configurations Do Not Increase Red Blood Cell (RBC) Agglutination

Anti-CD47 antibodies were shown to induce transient anemia in vivo. This induction of anemia is associated with red blood cell (RBC) agglutination in vitro. To determine whether the dimerized multivalent SIRPα fusion polypeptides V1, V2, and V3 configurations increased RBC agglutination relative to dimerized CV1-G4, in vitro RBC agglutinations were performed. Unexpectedly, V1, V2, and V3 configurations exhibited similar levels of RBC agglutination to CV1-G4, with no observable difference in agglutination due to the multivalency of the SIRPα fusion polypeptides (FIG. 5 and FIG. 6), under a range of concentrations tested (0.3 nM-267 nM).

Protein aggregation in biotherapeutics has been identified to increase immunogenicity, leading to immune-mediated adverse effects, enhanced drug clearance rates, and direct blockage of therapeutic function. The multivalent SIRPα fusion polypeptides, V1, V2, and V3, configurations showed no detectable protein aggregation (FIGS. 7A-7C). These results together with the significantly reduced RBC agglutination observed in vitro (FIG. 5 and FIG. 6) demonstrate that V1, V2, and V3 configurations have favorable safety profiles, as assessed in vitro. At the same time, the multivalent SIRPα fusion polypeptides V1, V2, and V3 configurations demonstrated enhanced potency in in vitro phagocytosis. Without being bound by theory, the increased size and avidity of V1, V2, and V3 configurations are also likely to improve the pharmacokinetics of these constructs relative to the smaller monovalent SIRPα fusion polypeptides (CV1-G4), thereby maximizing in vivo target cell uptake.

1. A multivalent signal-regulatory protein α (SIRPα) fusion polypeptide comprising at least two SIRPα domains and at least one Fc domain, wherein the SIRPα domains and Fc domains are in a ratio of at least two to one.

2. The multivalent SIRPα fusion polypeptide of claim 1, wherein the SIRPα domains and Fc domains are in a ratio of at least three to one.

3. The multivalent SIRPα fusion polypeptide of any one of claims 1-2, wherein two copies of the multivalent SIRPα fusion polypeptide form a dimer.

4. The multivalent SIRPα fusion polypeptide of any one of claims 1-3, wherein one or more SIRPα domains comprise the amino acid SEQ ID NO: 1 or an amino acid sequence with at least 70%, at least 75%, at least 80%, at least 85%, at least 90%, at least 95%, at least 96%, at least 97%, at least 98%, or at least 99% sequence identity thereto.

5. The multivalent SIRPα fusion polypeptide of any one of claims 1-3, wherein one or more SIRPα domains comprise one or more of the following mutations relative to SEQ ID NO: 1: V6I, S14L, S20T, I22T, H24R, V27I, I31F, A45G, E47V, K53R, E54Q, H56P, S66T, E70N, S77R, V92I, and/or a duplication of the D100 residue.

6. The multivalent SIRPα fusion polypeptide of any one of claims 1-4, wherein one or more SIRPα domains comprise the amino acid SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

7. The multivalent SIRPα fusion polypeptide of any one of claims 1-6, wherein the multivalent SIRPα fusion polypeptide comprises at least two SIRPα domains at the N terminus of the Fc domain.

8. The multivalent SIRPα fusion polypeptide of any one of claims 1-7, wherein the multivalent SIRPα fusion polypeptide comprises at least two SIRPα domains at the C terminus of the domain.

9. The multivalent SIRPα fusion polypeptide of any one of claims 1-8, wherein the multivalent SIRPα fusion polypeptide comprises at least one SIRPα domain at the C terminus and at least one SIRPα domain at the N terminus of the Fc domain.

10. The multivalent SIRPα fusion polypeptide of any one of claims 1-9, wherein the multivalent SIRPα fusion polypeptide comprises one or more constant heavy chain 1 (CH1) of an antibody.

11. The multivalent SIRPα fusion polypeptide of any one of claims 1-10, wherein the multivalent SIRPα fusion polypeptide comprises one or more constant light chains (CL) of an antibody.

12. The multivalent SIRPα fusion polypeptide of any one of claims 1-11, wherein the multivalent SIRPα fusion polypeptide comprises a hinge domain.

13. The multivalent SIRPα fusion polypeptide of any one of claims 1-12, wherein the multivalent SIRPα fusion polypeptide comprises a peptide linker of 6 to 16 amino acids in length.

14. The multivalent SIRPα fusion polypeptide of any one of claims 1-12, wherein the multivalent SIRPα fusion polypeptide comprises a light chain and a light chain, wherein the light chain comprises a CL and a SIRPα domain and the heavy chain comprises a CH1 and a SIRPα domain at the N terminus.

15. The multivalent SIRPα fusion polypeptide of any one of claims 1-12, wherein the multivalent SIRPα fusion polypeptide comprises a light chain and a light chain, wherein the light chain comprises a CL and a SIRPα domain and the heavy chain comprises a CH1 and a SIRPα domain at the N terminus and a SIRPα domain at the C terminus.

16. The multivalent SIRPα fusion polypeptide of any one of claims 1-12, wherein the multivalent SIRPα fusion polypeptide comprises two SIRPα domains at the N terminus and one SIRPα domain at the C terminus.

17. The multivalent SIRPα fusion polypeptide of any one of claims 1-12, wherein the multivalent SIRPα fusion polypeptide comprises a light chain and a light chain, wherein the light chain comprises a CL and a SIRPα domain and the heavy chain comprises a CH1 at the N terminus and a SIRPα domain at the C terminus.

18. The multivalent SIRPα fusion polypeptide of any one of claims 1-17, wherein the Fc domain is a human IgG1, IgG2, IgG3, or IgG4 Fc region.

19. The multivalent SIRPα fusion polypeptide of any one of claims 1-18, wherein the Fc domain comprises at least one mutation relative to a wild-type human IgG1, IgG2, IgG3, or IgG4 Fc region.

20. The multivalent SIRPα fusion polypeptide of claim 19, wherein the at least one mutation comprises one or more of M252Y, S254T, and/or T256E relative to an Fc of IgG1.

21. The multivalent SIRPα fusion polypeptide of any one of claims 1-18, wherein the SIRPα domain comprises either of SEQ ID NOS: 1 or 2, or an amino acid sequence with at least 70%, at least 75%, at least 80%, at least 85%, at least 90%, at least 95%, at least 96%, at least 97%, at least 98%, or at least 99% sequence identity thereto; and the Fc domain comprises any of SEQ ID NOS: 6-9, or an amino acid sequence with at least 70%, at least 75%, at least 80%, at least 85%, at least 90%, at least 95%, at least 96%, at least 97%, at least 98%, or at least 99% sequence identity thereto.

22. The multivalent SIRPα fusion polypeptide of any of claims 10-21, wherein the CH1 comprises either of SEQ ID NOS: 19 or 20, or an amino acid sequence with at least 70%, at least 75%, at least 80%, at least 85%, at least 90%, at least 95%, at least 96%, at least 97%, at least 98%, or at least 99% sequence identity thereto.

23. The multivalent SIRPα fusion polypeptide of any of claims 11-22, wherein the CL comprises a sequence of SEQ ID NO: 21, or an amino acid sequence with at least 70%, at least 75%, at least 80%, at least 85%, at least 90%, at least 95%, at least 96%, at least 97%, at least 98%, or at least 99% sequence identity thereto.

24. The multivalent SIRPα fusion polypeptides of any one of claims 1-18, comprising any of SEQ ID NOS: 10-18, or an amino acid sequence with at least 70%, at least 75%, at least 80%, at least 85%, at least 90%, at least 95%, at least 96%, at least 97%, at least 98%, or at least 99% sequence identity thereto.

25. The multivalent SIRPα fusion polypeptides of any one of claims 1-18, comprising: SEQ ID NO: 12 or an amino acid sequence with at least 70%, at least 75%, at least 80%, at least 85%, at least 90%, at least 95%, at least 96%, at least 97%, at least 98%, or at least 99% sequence identity thereto; and SEQ ID NO: 13, or an amino acid sequence with at least 70%, at least 75%, at least 80%, at least 85%, at least 90%, at least 95%, at least 96%, at least 97%, at least 98%, or at least 99% sequence identity thereto.

26. The multivalent SIRPα fusion polypeptides of any one of claims 1-18, comprising: SEQ ID NO: 14 or an amino acid sequence with at least 70%, at least 75%, at least 80%, at least 85%, at least 90%, at least 95%, at least 96%, at least 97%, at least 98%, or at least 99% sequence identity thereto; and SEQ ID NO: 15, or an amino acid sequence with at least 70%, at least 75%, at least 80%, at least 85%, at least 90%, at least 95%, at least 96%, at least 97%, at least 98%, or at least 99% sequence identity thereto.

27. The multivalent SIRPα fusion polypeptides of any one of claims 1-18, comprising: SEQ ID NO: 17 or an amino acid sequence with at least 70%, at least 75%, at least 80%, at least 85%, at least 90%, at least 95%, at least 96%, at least 97%, at least 98%, or at least 99% sequence identity thereto; and SEQ ID NO: 18, or an amino acid sequence with at least 70%, at least 75%, at least 80%, at least 85%, at least 90%, at least 95%, at least 96%, at least 97%, at least 98%, or at least 99% sequence identity thereto.

28. The multivalent SIRPα fusion polypeptide of any one of claims 1-27, wherein the multivalent SIRPα fusion polypeptide has a higher binding affinity to a CD47 protein.

29. The multivalent SIRPα fusion polypeptide of claim 28, wherein the CD47 protein is a human or mouse CD47 protein.

30. The multivalent SIRPα fusion polypeptide of any one of claims 1-29, wherein the multivalent SIRPα fusion polypeptide exhibits a higher binding strength to a cell expressing a CD47 protein.

31. The multivalent SIRPα fusion polypeptide of any one of claims 1-30, wherein the multivalent SIRPα fusion polypeptide exhibits a slower blood clearance.

32. The multivalent SIRPα fusion polypeptide of any one of claims 1-31, wherein the multivalent SIRPα fusion polypeptide is effective at a lower dose in a subject.

33. The multivalent SIRPα fusion polypeptide of any claims 1-32, wherein the multivalent SIRPα fusion polypeptide is effective at a lower dosage frequency.

34. The multivalent SIRPα fusion polypeptide of any one of claims 1-33, wherein the multivalent SIRPα fusion polypeptide provides increased phagocytosis in a phagocytosis assay.

35. The multivalent SIRPα fusion polypeptide of any one of claims 1-34, wherein the multivalent SIRPα fusion polypeptide is associated with comparable red blood cell in vitro agglutination.

36. The multivalent SIRPα fusion polypeptide of any one of claims 1-35, wherein the multivalent SIRPα fusion polypeptide exhibits comparable expression purity.

37. A method of treating a disease or disorder in a subject in need thereof, comprising administering a multivalent SIRPα fusion polypeptide of any one of claims 1-36 to the subject.

38. The method of claim 37, wherein the disease or disorder is selected from the group consisting of a cardiovascular disease, a cancer, fibrosis, an infectious disease, a hematological disease, and a neurological disease.

39. The method of claim 38, wherein the disease is a cardiovascular disease.

40. The method of any of claims 37-39, wherein the multivalent SIRPα fusion polypeptide is administered at a lower dose relative to a monovalent SIRPα fusion polypeptide.

41. The method of any of claims 37-40, wherein the multivalent SIRPα fusion polypeptide is administered at a lower dosage frequency relative to a monovalent SIRPα fusion polypeptide.

42. The method of any of claims 37-41, wherein the multivalent SIRPα fusion polypeptide is administered subcutaneously.

43. The method of any of claims 37-42, wherein the multivalent SIRPα fusion polypeptide is administered in combination with an anti-CD20 antibody.

44. The method of any of claims 37-42, wherein the multivalent SIRPα fusion polypeptide is administered in combination with an anti-CD47 antibody.

45. A method of inducing increased phagocytosis in macrophages comprising contacting a population of macrophages with a multivalent SIRPα fusion polypeptide of any one of claims 1-36.

46. The method of claim 45, wherein the macrophage is a human macrophage.

47. A method of imaging CD47 expression in a subject or a cell comprising administering to a subject or a cell a multivalent SIRPα fusion polypeptide of any one of claims 1-36.

48. A nucleotide encoding the multivalent SIRPα fusion polypeptide of any one of claims 1-36.
